(19) United States
(12) Patent Application Publication
(10) Pub. No.: US 2023/0404859 A1
(43) Pub. Date: Dec. 21, 2023

(54) PH-SENSITIVE MICROCAPSULE FOR DENTAL USE

(71) Applicant: 3M INNOVATIVE PROPERTIES COMPANY, St. Paul, MN (US)

(72) Inventors: Kai U. Claussen, Duesseldorf (DE); Hassan Sahouani, Hastings, MN (US); Silke D. Mechernich, Duesseldorf (DE)

(21) Appl. No.: 18/037,642

(22) PCT Filed: Nov. 16, 2021
(86) PCT No.: PCT/IB2021/060608

§ 371 Date: May 18, 2023

(30) Foreign Application Priority Data
Dec. 4, 2020 (EP) 20211733.9

(51) Int. Cl. A61K 6/15; A61K 6/61; A61K 6/70

(52) U.S. Cl. CPC A61K 6/15; A61K 6/61; A61K 6/70

(57) ABSTRACT

The invention relates to a dental composition comprising a polymerizable component not comprising an acidic moiety, and microcapsules comprising a first component of a non water-soluble redox-initiator system, and a pH-sensitive inorganic component, the microcapsules not comprising a polymeric material. The invention also relates to the use of certain microcapsules for preparing a curable dental composition and to a process of curing a dental composition, as well as a device for storing the dental composition.

### FIELD OF THE INVENTION

The invention relates to a dental composition comprising polymerizable components and microcapsules comprising a pH-sensitive component and a component of a redox-initiator system.

### BACKGROUND The use of microcapsules for storing components such as components of redox initiator systems is generally known.

U.S. Pat. No. 5,154,762 (Mitra et al.) relates to a water-containing, ionically-hardenable, photocurable, ethylenically-unsaturated dental cement, comprising a) finely-divided acid-reactive filler, b) water-miscible acidic polymer, c) photo-initiator, d) water-soluble reducing agent, and water-soluble oxidizing agent, wherein the reducing agent or oxidizing agent may be contained in microcapsules. As an encapsulant a medically acceptable polymer and film former is suggested.

WO 2019/234661 A1 (3M IPC) describes a two part hardenable dental composition comprising a first part comprising a composition comprising a liquid material and an encapsulated material which comprises a basic core material and an inorganic shell material comprising a metal oxide surrounding the core and a second part comprising a composition comprising a liquid material. It is stated that the encapsulated basic material is typically not a reducing agent of a redox curing system as this would delay the redox curing reaction.

US 2020/368117 A1 (Claussen et al) relates to a microcapsule comprising a hollow or porous core being composed of a polymeric material and containing a component of a redox-initiator system, and a shell being composed of a pH-sensitive material. It is outlined that the crosslinked matrix of the polymeric material is sufficiently stable to withstand shear forces which occur during mixing processes.

This technology is particularly useful for preparing powder compositions. However, compared to paste/paste systems powder compositions are easier to prepare and stabilize due to the physical separation of the powder components to be mixed.

In contrast thereto, pasty compositions are typically manufactured by kneading processes where high shear forces are applied onto the microcapsules.

The microcapsules described in the art, in particular those suggested for storing components of a redox-initiator system, are typically not sufficiently stable to survive such high shear forces.

On the other hand, when mixing paste/paste compositions the applied mixing forces are often not sufficiently strong enough for breaking the microcapsules to enable the release of the active reagents.

Therefore, the technology suggested for producing powder compositions can typically not be used for paste/paste compositions.

Other references which describe the use and production of microcapsules are:

US 2016/0088836 A1 (Sahouani et al.) describes polymeric composite particle which can be used for the storage and delivery of biologically active agents. The polymeric composite particles contain a porous polymeric core and a coating layer around the polymeric core.

WO 2007/013794 A1 (Stichting Gronignen Centre for Drug Research) describes a pH-controlled pulsatile release system comprising a core surrounded by a coating layer, wherein the core comprises an active substance and the coating layer comprises a pH-sensitive coating material wherein a swellable agent is embedded. As swellable agent sodium starch glycolate is suggested.

U.S. Pat. No. 6,022,501 (Dexter et al.) relates to pH-sensitive microcapsules comprising a water-immiscible active ingredient within a shell wall wherein said shall wall has free carboxylic acid groups incorporated therein.

Jun Wang et al. describe in J. Phys. Chem C 2010, 114, 18940-18945 calcium carbonate/carboxymethyl chitosan hybrid microspheres for drug delivery, in particular for the delivery of a water-soluble anticancer drug.

Sukhorukov et al. describe in J. Mater. Chem., 2004, 14, 2073-2081 porous calcium carbonate microparticles as templates for encapsulation of bioactive compounds, in particular biomacromolecules.

Yu et al. describes in Applied Energy 114 (2014), 632-643 a process of mircroencapsulation of n-octadecane phase change material with calcium carbonate shell for enhancement of thermal conductivity and serving durability.

### SUMMARY OF INVENTION

There is a desire for microcapsules which can be used for producing storage-stable pasty compositions.

There is also a desire for a microcapsule which is sufficiently mechanically stable allowing the microcapsule to be processes in a kneading apparatus and allows a release on demand of a reagent or component stored in the microcapsule.

Further, the release on demand of the component to be released should be sufficiently fast.

It would also be desirable, if the setting behaviour of the compositions is not negatively affected.

If possible, the microcapsule should also allow for a high loading of the component to be stored in the microcapsule.

One or more of these objects are addressed by the microcapsules and related processes described in the claims and the present text.

In one embodiment the invention features a dental composition comprising a polymerizable component not comprising an acidic moiety, and microcapsules comprising a non water-soluble first component of a redox-initiator system, and a pH-sensitive inorganic component, the microcapsules not comprising a polymeric material the pH-sensitive inorganic component preferably forming a shell around the core which is comprised of the non-water soluble first component.

The invention is also related to the use of the microcapsules described in the present text for preparing a curable dental composition.

In addition, the invention is directed to a process of curing the dental composition described in the present text, the process comprising the step of combining the Base Paste and the Catalyst Paste, wherein the acidic component comes in contact with the microcapsule comprising the first component of the redox-initiator system and reacts with the pH-sensitive inorganic component of the microcapsule, wherein the pH-sensitive inorganic component of the microcapsule is dissolved and the non water-soluble first component of the redox-initiator system is released.

The invention is also directed to a device for storing the dental composition.

Further, the invention is directed to a kit of parts comprising the dental composition described in the present text and the following parts alone or in combination: a dental milling block for machining a dental restoration, a dental adhesive.

Moreover, the invention features a dental composition as described in the present text for use in a method or restoring a dental tooth in the mouth of a patient, the method comprising the steps of providing the dental composition in the form of a kit of parts comprising a Base Paste and a Catalyst Paste, mixing the Base Paste and the Catalyst Paste, applying the mixture to the surface of a prepared tooth to be restored.

Unless defined differently, for this description the following terms shall have the given meaning:

An “initiator” is a substance being able to initiate a chemical reaction, preferably via a free radical reaction. The initiator can be a single compound or can comprise more than one component, such as a combination of a sensitizing agent with a reducing agent. Depending on the reaction conditions chosen (e.g. pH-value>7 or pH-value<7) different initiators can be preferred.

A “redox-initiator system” is defined as the combination of reducing agent(s) and oxidizing agent(s). If present, transition metal component(s) are also regarded as components of the redox-initiator system.

As used herein, “hardening” or “curing” are used interchangeably and refer to polymerization and/or crosslinking reactions including, for example, photopolymerization reactions and chemical polymerization techniques (e. g., ionic reactions or chemical reactions forming radicals, effective to polymerize ethylenically unsaturated compounds) involving one or more materials included in the composition.

“Dental article” means an article which is to be used in the dental field, especially as or for producing a dental restoration. A dental article has typically two different surface portions, an outer surface and an inner surface. The outer surface is the surface which is typically not in permanent contact with the surface of a tooth. In contrast thereto, the inner surface is the surface which is used for attaching or fixing the dental article to a tooth. If the dental article has the shape of a dental crown, the inner surface has typically a concave shape, whereas the outer surface has typically a convex shape. A dental article should not contain components which are detrimental to the patient's health and thus free of hazardous and toxic components being able to migrate out of the dental or orthodontic article.

A “dental composition” or a “composition for dental use” or a “composition to be used in the dental field” is any composition which can be used in the dental field. In this respect, the composition should be not detrimental to the patients' health and thus be free of hazardous and toxic components being able to migrate out of the composition. Examples of dental compositions include permanent and temporary crown and bridge materials, artificial crowns, anterior or posterior filling materials, adhesives, mill blanks, lab materials, luting agents and orthodontic devices. Dental compositions are typically hardenable compositions, which can be hardened at ambient conditions, including a temperature range of 15 to 50° C. or from 20 to 40° C. within a time frame of 30 min or 20 min or 10 min. Higher temperatures are not recommended as they might cause pain to the patient and may be detrimental to the patient's health. Dental compositions are typically provided to the practitioner in comparable small volumes, that is volumes in the range of 0.1 to 100 ml or 0.5 to 50 ml or 1 to 30 ml. Thus, the storage volume of useful packaging devices is within these ranges. “Dental restoration” means dental articles which are used for restoring a tooth to be treated.

Examples of dental restorations include crowns, bridges, inlays, onlays, veneers, facings, copings, crown and bridged framework, and parts thereof.

The term “compound” or “component” is a chemical substance which has a particular molecular identity or is made of a mixture of such substances, e.g., polymeric substances. A “polymerizable component” is any component which can be cured or solidified e.g. by heating to cause polymerization or chemical crosslinking, or e.g. by radiation-induced polymerization or crosslinking, or e.g. using a redox initiator or by any other radical forming process. A radically polymerizable component may contain only one, two, three or more radically polymerizable groups. Typical examples of radically polymerizable groups include unsaturated carbon groups, such as a vinyl group being present e.g. in a (methyl)acrylate group.

A “monomer” is any chemical substance which can be characterized by a chemical formula, bearing radically polymerizable unsaturated groups (including (meth)acrylate groups) which can be polymerized to oligomers or polymers thereby increasing the molecular weight. The molecular weight of monomers can usually simply be calculated based on the chemical formula given. “Polymer” or “polymeric material” are used interchangeably to refer to a homopolymer, copolymer, terpolymer etc.

A “derivative” or “structural analogue” is a chemical compound showing a chemical structure closely related to the corresponding reference compound and containing all featured structural elements of the corresponding reference compound but having small modifications like bearing additional chemical groups like e.g. alkyl moieties, Br, Cl, or F or not bearing chemical groups like e.g. alkyl moieties in comparison to the corresponding reference compound. That is, a derivative is a structural analogue of the reference compound. A derivative of a chemical compound is a compound comprising the chemical structure of said chemical compound.

As used herein, “(meth)acryl” is a shorthand term referring to “acryl” and/or “methacryl”. For example, a “(meth) acryloxy” group is a shorthand term referring to either an acryloxy group (i.e., CH₂=CH—(O)—O—) and/or a methacryloxy group (i.e., CH₂=C(CH₃)—C(O)—O—).

A component comprising an “ascorbic acid moiety” is a component comprising the following structural element:

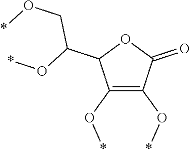 [SMILES: *OCC(O*)C1OC(=O)C(O*)=C1O*]

wherein the symbol “*” indicates a connection to another chemical moiety or atom.

A “particle” means a substance being a solid having a shape which can be geometrically determined. The shape can be regular or irregular. Particles can typically be analysed with respect to e.g. particle size and particle size distribution.

The particle size (d50) of a powder can be obtained from the cumulative curve of the grain size distribution. Respective measurements can be done using commercially available granulometers (e.g. Malvern Mastersizer 2000). “D” represents the diameter of powder particles and “50” refers to the volume percentage of the particles. Sometimes, the 50% is also expressed as “0.5”. For example, “(d50) =1 μm” means that 50% of the particles have a size of 1 μm or less.

“Ambient conditions” mean the conditions which the composition described in the present text is usually subjected to during storage and handling. Ambient conditions may, for example, be a pressure of 900 to 1,100 mbar, a temperature of 10 to 40° C. and a relative humidity of 10 to 100%. In the laboratory ambient conditions are typically adjusted to 20 to 25° C. and 1,000 to 1,025 mbar (at maritime level).

As used herein, “a”, “an”, “the”, “at least one” and “one or more” are used interchangeably. Also herein, the recitations of numerical ranges by endpoints include all numbers subsumed within that range (e.g., 1 to 5 includes 1, 1.5, 2, 2.75, 3, 3.80, 4, 5, etc.). Adding an “(s)” to a term means that the term should include the singular and plural form. E.g.

the term “additive(s)” means one additive and more additives (e.g. 2, 3, 4, etc.).

Unless otherwise indicated, all numbers expressing quantities of ingredients, measurement of physical properties such as described below and so forth used in the specification and claims are to be understood as being modified in all instances by the term “about”.

The terms “comprise” or “contain” and variations thereof do not have a limiting meaning where these terms appear in the description and claims. “Consisting essentially of” means that specific further components can be present, namely those which do not materially affect the essential characteristic of the article or composition. “Consisting of” means that no further components should be present. The term “comprise” shall include also the terms “consist essentially of” and “consists of”.

A composition is “essentially or substantially free of” a certain component, if the composition does not contain said component as an essential feature. Thus, said component is not willfully added to the composition either as such or in combination with other components or ingredient of other components. A composition being essentially free of a certain component usually does not contain that component at all. However, sometimes the presence of a small amount of the said component is not avoidable e.g. due to impurities contained in the raw materials used.

### DETAILED DESCRIPTION

It has been found that the composition described in the text has a couple of advantageous properties.

The microcapsules are sufficiently mechanically stable and survive shear forces which typically occur during production processes involving a kneading step, e.g. when preparing pasty compositions.

The microcapsules contain a component which is pH-sensitive, that is a component which undergoes a chemical reaction, in particular an acid/base reaction, if the pH is changed. Particularly, pH-sensitive means sensitive to an acidic environment.

Due to this property, the microcapsules described in the present text can also be regarded pH-sensitive microcapsules.

An example of a pH-sensitive component is a component which dissolves upon contact to an acidic component or produces gas upon exposure to an acidic component. Such a component is particularly useful as it enables a fast release of the component to be released from the microcapsule demand. If gas is produced, the gas helps to widen, perforate and/or break up the microcapsule.

This can be advantageous for e.g. redox-initiator systems contained in a dental two-part paste/paste composition that contain an acidic component.

Thus, the microcapsules described in the present text can help to overcome challenges associated e.g. with the production of redox-curable paste/paste compositions.

For producing storage-stable paste/paste compositions which contain a redox-initiator system a fast release of the redox-initiator components is often desired to ensure the hardening of the curable composition within an appropriate time.

Compared to microcapsules described in the prior art, the microcapsule described in the present text allows for a comparable high loading of the component to be released as this component is directly encapsulated without the need for additional shells or layers.

Thus, for providing a sufficient amount of component to be released, a lower amount of microcapsules is needed. This can be advantageous in applications where the amount of shell material used might have an impact. E.g. in a dental curable composition the presence of a too high amount of shell material might have a negative impact on the aesthetics of the cured composition.

The invention relates to a dental composition comprising a polymerizable component and a microcapsule comprising a non water-soluble first component of a redox-initiator system.

The microcapsules can typically be characterized by the following features alone or in combination: diameter: 1 to 200 μm or 1 to 100 μm or 5 to 100 μm or 5 to 50 um or 5 to 25 um; being mechanically stable.

Using microcapsules have a diameter in the range of 1 to 100 μm is sometimes preferred. It was also found that by using components having a small particle size, the reaction between an acidic component and the pH-sensitive component of the microcapsule can be improved as the reaction surface is increased.

The shape and diameter can be evaluated by microscopy, in particular by scanning electron microscopy (SEM). If desired, the diameter and particle size distribution can be determined by light scattering.

A microcapsule is mechanically stable, if it is able to survive high shear forces, which typically occur during preparing pastes in a mixing or kneading machine. A suitable test is described in the example section.

Mechanical stability can be obtained, e.g. if the microcapsule mainly comprises an inorganic component and the presence of a polymeric material is avoided.

The microcapsule comprises a pH-sensitive inorganic component. Without wishing to be bound to a particular theory, using an inorganic component is considered beneficial as these microcapsules tend to be more mechanically stable than microcapsules comprising or consisting essentially of a polymeric material.

The pH-sensitive inorganic component forms a shell around the core which is comprised of the non-water soluble first component of a redox-initiator system.

The presence of a pH-sensitive inorganic component helps to facilitate the release of the component to be released and stored in the microcapsule, particularly if the microcapsule capsule is brought in contact with an acidic or basic environment.

The pH-sensitive inorganic component typically comprises an anion and a multivalent cation.

The anion is typically selected from carbonate, hydrogen carbonate, phosphate, nitrate and sulphate.

The multivalent cation is typically selected from ions of Mg, Ca, Sr, Ba, Al, and Zn.

Examples of pH-sensitive inorganic components include MgCO₃, CaCO₃, ZnCO₃, Ca(HCO₃)₂, CaSO₄, and MgSO₄.

The release of this component can be even further improved, if the pH-sensitive inorganic component produces gas upon contact with an acidic environment.

If such a microcapsule is brought in contact with an acidic environment, not only the pH-sensitive component is broken-up or dissolved, but the break-up is accelerated by the gas which is produced.

A component which produce gas upon exposure to an acid typically comprising a moiety selected from carbonate or hydrogen carbonate. The gas which is produced is typically CO₂.

Examples of components which produce gas upon exposure to an acid include particularly earth alkali metal (e.g. Mg, Ca) carbonates or hydrogen carbonates as well as Zn carbonate and hydrogen carbonates such as MgCO₃, CaCO₃, ZnCO₃, and mixtures thereof.

The use of the following components is sometimes preferred as they have been proven to be very effective for the desired use: CaCO₃, Ca(HCO₃)₂.

If the microcapsules are to be used in the medical or dental field, the components should be sufficiently biocompatible and essentially non-toxic in the amount used.

The microcapsule contains a component to be released. The component to be released is contained in the microcapsule, particularly in the core of the microcapsule.

Any kind of component to be released can be stored in the microcapsule which does not negatively interact with the pH-sensitive inorganic material of the microcapsule.

According to one embodiment the microcapsule contains a first component of a redox-initiator system. Such a component is sometimes referred to as active agent.

The first component of the redox-initiator system is non water-soluble. Non water-soluble means that the solubility of the component is less than 0.1 g/100 ml water or less than 0.05 g/100 ml water (23° C.).

A suitable method for determining the solubility of a component in water is given in the example section.

Further, the first component of the redox-initiator system is typically provided as a solid component. That is, the first component is provided in a particulate form.

The first component of the redox-initiator system is able to form a suspension in water upon stirring.

A redox-initiator system typically comprises oxidizing agent(s) and reducing agent (s) and sometimes transition metal(s).

According to one embodiment, the microcapsule contains a non water-soluble oxidizing agent.

The nature and structure of the oxidizing agent is not particularly limited unless the desired result cannot be achieved.

Suitable oxidizing agents include non water-soluble organic peroxide and persulfate component(s) and mixtures thereof

Generally, all non water-soluble peroxide(s), in particular organic peroxides, which can be incorporated or absorbed by the microcapsules can be used.

Organic peroxides which can be used include hydroperoxide(s), ketone peroxide(s), diacyl peroxide(s), dialkyl peroxide(s), peroxyketal(s), peroxyester(s) and peroxydicarbonate(s).

Di-peroxides, which can be used include di-peroxides comprising the moiety R₁—O—O—R₂—O—O—R₃, with R₁ and R₃ being independently selected from H, alkyl (e.g. C₁ to C₆), branched alkyl (e.g. C₁ to C₆), cycloalkyl (e.g. C₅ to C₁₀), alkylaryl (e.g. C₇ to C₁₂) or aryl (e.g. C₆ to C₁₂) and R₂ being selected from alkyl (e.g. (C₁ to C₆) or branched alkyl (e.g. C₁to C₆).

Examples of ketone peroxides include methyl ethyl ketone peroxide, methyl isobutyl ketone peroxide, methyl cyclohexanone peroxide, and cyclohexanone peroxide.

Examples of peroxyesters include cumylperoxyneodecanoate, t-butyl peroxypivarate, t-butyl peroxyneodecanoate, 2,2,4-trimethylpentylperoxy-2-ethyl hexanoate, t-amylperoxy-2-ethyl hexanoate, t-butylperoxy-2-ethyl hexanoate, di-t-butylperoxy isophthalate, di-t-butylperoxy hexahydroterephthalate, t-butylperoxy-3,3,5-trimethylhexanoate, t-butylperoxy acetate, t-butylperoxy benzoate and t-butylperoxymaleic acid.

Examples of peroxidicarbonates include di-3-methoxy peroxidicarbonate, di-2-ethylhexyl peroxydicarbonate, bis(4-t-butylcyclohexyl)peroxidicarbonate, diisopropyl-1-peroxydicarbonate, di-n-propyl peroxidicarbonate, di-2-ethoxyethyl-peroxidicarbonate, and diallyl peroxidicarbonate.

Examples of diacyl peroxides include acetyl peroxide, benzoyl peroxide, decanoyl peroxide, 3,3,5-trimethylhexanoyl peroxide, 2,4-dichlorobenzoyl peroxide and lauroylperoxide.

Examples of dialkyl peroxides include di-t-butyl peroxide, dicumylperoxide, t-butylcumyl peroxide, 2,5-dimethyl-2,5-di(t-butylperpoxy)hexane, 1,3-bis(t-butylperoxyisopropyl)benzene and 2,5-dimethyl-2,5-di(t-butylperoxy)-3-hexane.

Examples of peroxyketals include 1,1-bis(t-butylperoxy)-3,3,5-trimethylcyclohexane, 1,1-bis(t-butylperoxy)cyclohexane, 2,2-bis(t-butylperoxy)butane, 2,2-bis(t-butylperoxy)octane and 4,4-bis(t-butylperoxy)valeric acid-n-butylester.

According to one embodiment, the organic peroxide is a hydroperoxide, in particular a hydroperoxide comprising the structural moiety R—O—O—H with R being (e.g. C₁ to C₂₀) alkyl, (e.g. C₃ to C₂₀) branched alkyl, (e.g. C₆ to C₁₂) cycloalkyl, (e.g. C₇ to C₂₀) alkylaryl or (e.g. C₆ to C₁₂) aryl.

Examples of suitable organic hydroperoxides include t-butyl hydroperoxide, t-amyl hydroperoxide, p-diisopropylbenzene hydroperoxide, cumene hydroperoxide, pinane hydroperoxide, p-methane hydroperoxide and 1,1,3,3-tetramethylbutyl hydroperoxide.

Suitable peroxodisulfate components and/or peroxodiphosphate components and/or mixtures thereof, which can be used include organic and/or inorganic components. Suitable examples include ammonium, sodium, and potassium peroxodisulfate components and/or peroxodiphosphate components. Sodium peroxodisulfate is sometimes preferred.

According to another embodiment, the microcapsule contains a non water-soluble reducing agent.

The nature and structure of the non water-soluble reducing agent is not particularly limited unless the desired result cannot be achieved.

Suitable reducing agents include organic and inorganic component(s) and mixtures thereof.

The reducing agent is typically a solid at ambient conditions (23° C.; 1013 hPa).

Reducing agents (s) which may be contained in the microcapsule include non water-soluble ascorbic acid component(s), tertiary amine component(s), sulfinate component(s), sulphite component(s), borane component(s), (thio)urea component(s), and (thio)barbituric acid component(s), saccharin and metal salts thereof.

Component(s) comprising an ascorbic acid moiety such as salts and esters of ascorbic acid, ethers, ketals, or acetals are sometimes preferred. These components are referred to as ascorbic acid components.

Suitable salts include the alkali metal and earth alkali metal salts like Na, K, Ca and mixtures thereof.

Esters of ascorbic acid include those, which are formed by reacting one or more of the hydroxyl functions of ascorbic acid with a carboxylic acid, in particular the C₂ to C₃₀ carboxylic acid.

Suitable examples of C₂ to C₃₀ carboxylic acids include the fatty acids, like caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid.

In particular preferred are these ascorbic acid components, which can be easily dissolved in or mixed with the remaining resin matrix comprising polymerizable components.

That is, using an ascorbic acid component having a hydrophobic moiety can sometimes be preferred. Suitable hydrophobic moieties include saturated and unsaturated aliphatic residues (e.g. C₂ to C₃₀ or C₁₂ to C₃₀). Those ascorbic acid components may also function as surface-active substances (substances having a so-called “head/tail structure”). Particularly preferred are sometimes ascorbyl palmitate, ascorbyl stearate, mixtures and salts thereof.

If the oxidizing agents(s) are brought in contact with the reducing agents(s), a redox-reaction typically starts. Such a redox-reaction is suitable to initiate the curing of curable components resulting in the crosslinking of the curable components.

If desired, the microcapsule can also be filled with other components in addition, for example dye(s), fluoride releasing agent(s). Suitable dye(s) and fluoride releasing agent(s) are described in the text below.

Typically, the content of the pH-sensitive component in the microcapsule is greater than the content of the first component of a redox-initiator system. The larger the particles of the first component of the redox-initiator system are, the lower the amount of the pH-sensitive typically is.

A ratio of the pH-sensitive component of the microcapsules to the first component of the redox-initiator system in a range of less than 20 to 1 or less than 10 to 1 or less than 5 to 1 or less than 3 to 1 with respect to weight was found to be useful.

Such a ratio was found to be beneficial as it provides a good balance between the amount of desired redox-initiator component to be released and the component which is used for producing the microcapsule, which it not always desired in the final composition. The microcapsule described in the present text allows for an incorporation or loading of the first component of the redox-initiator system in an amount of up to 50 or up to 40 wt. % with respect to the weight of the microcapsule.

If desired, the mass or amount of the shell can be calculated using the following formula:

D=dₚ+2Shell thickness

VₛₕₑₗₗVₜₒₜ=Vₚ

(Vₛₕₑₗₗ/V_{g})=(Vₜₒₜ/Vₚ)−1=(D³/dₚ³)−1

ρₛₕₑₗₗ=mₛₕₑₗₗ/Vₛₕₑₗₗ

ρₚ=mₚ/Vₚ

mₛₕₑₗₗ=mₚ(ρₛₕₑₗₗ/ρₚ)[(D³/dₚ³)−1]

D being the diameter of the microcapsule, dₚ being the diameter of the particle contained in the microcapsule, mₛₕₑₗₗ being the mass of the shell, ρₛₕₑₗₗ being the density of the shell material, ρₚ being the density of the core particle, Vₛₕₑₗₗ being the volume of the shell, Vₜₒₜ being the total volume of the microcapsule, Vₚ being the volume of the particles contained in the microcapsule.

The microcapsules containing the first component of a redox-initiator system are present in the dental composition in an amount effective to initiate the curing of the polymerizable components in the presence of the second component of the redox-initiator system.

The dental composition typically contains the microcapsules in an amount of 0.05 to 10 wt. % or to 5 wt. % or 0.2 to 3 wt. % or 0.3 to 2 wt. % with respect to the dental composition.

The microcapsule described in the present text can be produced as follows:

A composition is provided comprising water, surfactant, non water-soluble component (e.g. first component of a redox-initiator system) and a water-soluble pH-sensitive inorganic component (comprising a mono-valent cation).

The composition is mixed.

The mixture (dispersion) is centrifugated and the sedimented part is separated.

The separated part is combined with an aqueous solution containing a water-soluble salt comprising a multi-valent (in particular di-valent) cation.

The obtained composition (dispersion) is mixed again, centrifugated, the sedimented part is separated, further purified as desired (e.g. by re-suspension in water and filtering), and dried.

The addition of a surfactant is typically advantageous as it may help to obtain a more homogeneous distribution of the components in the mixture.

As the non water-soluble component is not soluble in water, it forms a suspension in water.

Without wishing to be bound to a particular theory, it is believed that the surfactant forms micelles containing the non water-soluble component of the redox-initiator system and thus stabilize these components in the aqueous solution or environment.

The nature of the surfactant is not particularly limited unless the desired effect cannot be achieved.

Suitable surfactants include ionic surfactants (e.g., sulfate, sulfonate, phosphate, carboxylate esters, quaternary ammonium salts), amphoteric surfactants (e.g., sultaines, betaines) and non-ionic surfactants, wherein non-ionic surfactants are preferred.

Non-ionic surfactants typically have covalently bonded oxygen-containing hydrophilic groups, which are bonded to hydrophobic parent structures. Compared to other surfactants, non-ionic surfactants often foam less strongly.

Examples of non-ionic surfactants which can be used include alkyl polyglucosides, fatty amine ethoxylates, fatty alcohol ethoxylates, fatty acid alkanolamides, castor oil ethoxylates, alcohol ethoxylates/propoxylates and blends thereof (e.g. blend of decyl and undecyl glucosides; APG™ 325, BASF).

If a surfactant is used, it is typically used in only a small amount, e.g. 2 to 10 wt. % or 5 to 8 wt. % with respect to the amount of the pH-sensitive component, which is formed during the production process.

The dental composition described in the present text comprises a polymerizable component.

A polymerizable component comprises a component with at least one or two polymerizable moieties such as a (meth)acrylate moiety.

The crosslinking or polymerization of the polymerizable component can be initiated by using a redox-initiator system.

The polymerizable component does not contain an acidic moiety.

The polymerizable component without an acidic moiety is typically a free-radically polymerizable material, including ethylenically unsaturated monomer, monomers or oligomers or polymers.

Suitable polymerizable component(s) without acidic moiety(s) can be characterized by the following formula:

AₙBAₘ

with A being an ethylenically unsaturated group, such as a (meth)acryl moiety,

B being selected from (i) linear or branched C₁ to C₁₂ alkyl, optionally substituted with other functional groups (e.g. halogenides (including Cl, Br, I), OH or mixtures thereof) (ii) C₆ to C₁₂ aryl, optionally substituted with other functional groups (e.g. halogenides, OH or mixtures thereof), or (iii) organic group having 4 to 20 carbon atoms bonded to one another by one or more ether, thioether, ester, thioester, thiocarbonyl, amide, urethane, carbonyl and/or sulfonyl linkages,

m, n being independently selected from 0, 1, 2, 3, 4, 5 or 6 with the proviso that n+m is greater 0, that is that at least one A group is present.

Such polymerizable materials include mono-, di- or poly-acrylates and methacrylates such as methyl acrylate, methyl methacrylate, ethyl (meth)acrylate, isopropyl (meth)acrylate, n-hexyl (meth)acrylate, stearyl (meth)acrylate, allyl (meth)acrylate, glycerol di(meth)acrylate, the diurethane dimethacrylate called UDMA (mixture of isomers, e.g. Rohm Plex 6661-0) being the reaction product of 2-hydroxyethyl methacrylate (HEMA) and 2,2,4-trimethylhexamethylene diisocyanate (TMDI), glycerol tri(meth)acrylate, ethyleneglycol di(meth)acrylate, diethyleneglycol di(meth)acrylate, triethyleneglycol di(meth)acrylate, 1,3-propanediol diacrylate, 1,3-propanediol dimethacrylate, trimethylolpropane tri(meth)acrylate, 1,2,4-butanetriol tri(meth)acrylate, 1,4-cyclohexanediol di(meth)acrylate, pentaerythritol tri(meth)acrylate, pentaerythritol tetraacrylate, pentaerythritol tetramethacrylate, sorbitol hexa(meth)acrylate, bis[1-(2-(meth)acryloxy)]-p-ethoxyphenyldimethylmethane, bis[1-(3 -methacryloxy-2-hydroxy)]-p-propoxyphenyldimethylmethane (BisGMA), bis[1-(3-acryloxy-2-hydroxy)]-p-propoxy-phenyldimethylmethane and trishydroxyethyl-isocyanurate trimethacrylate; the bis-acrylates and bis-methacrylates of polyethylene glycols of molecular weight 200-500, copolymerizable mixtures of acrylated monomers (see e.g. U.S. Pat. No. 4,652,274), and acrylated oligomers (see e.g. U.S. Pat. No. 4,642,126); and vinyl compounds such as styrene, diallyl phthalate, divinyl succinate, divinyl adipate and divinylphthalate; polyfunctional (meth)acrylates comprising urethane, urea or amide groups. Mixtures of two or more of these free radically polymerizable materials can be used if desired.

Further polymerizable components which may be present include di(meth)acrylates of ethoxylated bis-phenol A, for example 2,2′-bis(4-(meth)acryloxytetraethoxyphenyl)propanes, urethane (meth)acrylates and (meth)acrylamides. The monomers used can furthermore be esters of [alpha]-cyanoacrylic acid, crotonic acid, cinnamic acid and sorbic acid.

It is also possible to use the methacrylic esters mentioned in EP 0 235 826, such as bis[3[4]-methacryl-oxymethyl-8(9)-tricyclo[5.2.1.0^{2,6}]decylmethyl triglycolate. Suitable are also 2,2-bis-4(3-methacryloxy-2-hydroxypropoxy)phenylpropane (Bis-GMA), 2,2-bis-4(3-methacryloxypropoxy)phenyl-propane, 7,7,9-trimethyl-4,13-dioxo-3,14-dioxa-5,12-diazahexadecane-1,16-dioxy dimethacrylate (UDMA), urethane (meth)acrylates and di(meth)acrylates of bishydroxymethyltricyclo-(5.2.1.0^{2,6})decane.

These ethylenically unsaturated monomers can be employed in the dental composition(s) either alone or in combination with the other ethylenically unsaturated monomers. In addition or besides those components, other hardenable components which can be added include oligomeric or polymeric compounds, such as polyester (meth)acrylates, polyether (meth)acrylates, polycarbonate (meth)acrylates and polyurethane (meth)acrylates. The molecular weight of these compounds is typically less than 20,000 g/mol, particularly less than 15,000 g/mol and in particular less than 10,000 g/mol.

The polymerizable component(s) without acidic moieties are typically present in the following amounts: lower amount: at least 5 or at least 10 or at least 20 wt. %; upper amount: utmost 65 or utmost or utmost 45 wt. %; range: 5 to 65 or 10 to 55 or 20 to 45 wt. %; wt. % with respect to the weight of the dental composition.

According to one embodiment, the dental composition described in the present text is provided in the form of a kit of parts comprising a Catalyst Paste and a Base Paste.

The Catalyst Paste comprises a polymerizable component not comprising an acidic moiety, the microcapsules comprising the first component of the redox-initiator system and optionally filler(s).

The Base Paste comprises an acidic component, a second component of the redox initiator system and optionally filler(s).

The Catalyst Paste and the Base Paste are separated from each other during storage.

The first and second component of the redox-initiator system together form an initiator system which is suitable to initiate the curing of the curable components being present in the Catalyst Paste or the Base Paste or in the Catalyst Paste and the Base Paste.

According to one embodiment, the first component of a redox-initiator system contained in the microcapsules is a reducing agent and the second component of the redox-initiator system is an oxidizing agent.

According to another embodiment, the first component of a redox-initiator system contained in the microcapsules is an oxidizing agent and the second component of the redox-initiator system is a reducing agent.

According to one embodiment, the kit of parts comprises two kinds of microcapsules, microcapsules containing a reducing agent and microcapsules containing an oxidizing agent.

The oxidizing and reducing agents include those described above.

The acidic component contained in the Base Paste is a component being suitable to interact with the pH-sensitive component of the microcapsule, such that the structure of the microcapsule is weakened (e.g. dissolved) enabling the first component of the redox-initiator system to migrate out of the microcapsule.

The nature and structure of the acidic component is not particularly limited unless the intended purpose cannot be achieved. Inorganic and organic acidic components can be used, as desired.

Inorganic acidic components which can be used include hydrochloric acid, sulfuric acid, phosphoric acid, mixtures thereof and its acidic salts.

Organic acidic components which can be used include monocarboxylic acids such as formic acid, acetic acid and benzoic acid and derivatives of these acids or dicarboxylic acids chosen from oxalic acid, malonic acid, succinic acid, adipic acid, pimelic acid, azelaic acid, sebacic acid, maleic acid, fumaric acid, sorbic acid, phthalic acid and terephthalic acid and derivatives of these acids or tricarboxylic acids chosen from hemimellitic acid, trimellitic acid, trimesic acid, agaric acid, citric acid, 1,2,3-propanetricarboxylic acid and derivatives of these acids or multicarboxylic acids chosen from the group consisting of pyromellitic acid and mellitic acid and derivatives of these acids or polycarboxylic acids chosen from polyacrylic acid and polymethacrylic acid and derivatives of these acids and mixtures thereof.

The acidic component can be characterized by the following features alone or in combination:
a) pKs value: equal to or below 5, equal to or below 4 or equal to or below 3.5 or equal to or below 3 or equal to or below 2;
b) comprising acidic moieties selected from sulfonic, sulfinic, phosphoric, phosphonic, phosphinic, or carboxylic moieties.

If desired, the acidic component may also comprise one or more polymerizable moieties, such as (meth)acrylate moieties. One or more polymerizable component(s) with acidic moiety(s) may be present, if desired.

The polymerizable components with acid moiety can typically be represented by the following formula

AₙBCₘ
with A being an ethylenically unsaturated group, such as a (meth)acryl moiety,

B being a spacer group, such as (i) linear or branched C₁ to C₁₂ alkyl, optionally substituted with other functional groups (e.g. halogenides (including Cl, Br, I), OH or mixtures thereof) (ii) C₆ to C₁₂ aryl, optionally substituted with other functional groups (e.g. halogenides, OH or mixtures thereof), (iii) organic group having 4 to 20 carbon atoms bonded to one another by one or more ether, thioether, ester, thioester, thiocarbonyl, amide, urethane, carbonyl and/or sulfonyl linkages, and

C being an acidic group, or precursor of an acidic group such as acid anhydride,

m, n being independently selected from 1, 2, 3, 4, 5 or 6,

wherein the acidic group comprises one or more carboxylic acid residues, such as —COOH or —CO—O—CO—, phosphoric acid residues, such as —O—P(O)(OH)OH, phosphonic acid residues, such as C—P(O)(OH)(OH), sulfonic acid residues, such as —SO₃H or sulfinic acid residues such as —SO₂H.

Examples of polymerizable components with acid moiety include, but are not limited to glycerol phosphate mono(meth)acrylate, glycerol phosphate di(meth)acrylate, hydroxyethyl (meth)acrylate (e.g., HEMA) phosphate, bis((meth)acryloxyethyl) phosphate, (meth)acryloxypropyl phosphate, bis((meth)-acryloxypropyl) phosphate, bis((meth)acryloxy)propyloxy phosphate, (meth)acryloxyhexyl phosphate, bis((meth)acryloxyhexyl) phosphate, (meth)acryloxyoctyl phosphate, bis((meth)acryloxyoctyl) phosphate, (meth)acryloxydecyl phosphate, bis((meth)acryloxydecyl) phosphate, caprolactone methacrylate phosphate, citric acid di- or tri-methacrylate, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like. Derivatives of these hardenable components bearing an acid moiety that can readily react e.g. with water to form the specific examples mentioned above, like acid halides or anhydrides are also contemplated.

Also monomers, oligomers, and polymers of unsaturated carboxylic acids such as (meth)acrylic acids, aromatic (meth)acrylated acids (e.g., methacrylated trimellitic acids), and anhydrides thereof can be used.

If present, the acidic component(s) are typically present in the following amounts: lower amount: at least 2 or at least 3 or at least 4 wt. %; upper amount: utmost 50 or utmost 40 or utmost 30 wt. %; range: 2 to 50 or 3 to 40 or 4 to 30 wt. %; wt. % with respect to the weight of the dental composition obtained if the Catalyst Paste and the Base Paste of the kit of parts were combined.

The dental composition or the respective kit of parts and the Catalyst Paste and/or the Base Paste thereof may contain further components, including filler(s), photo initiator(s) and additives including fluoride release agent(s), stabilizer(s), colorant(s), dye(s), pigment(s), and other additive(s).

The amounts and types of each ingredient in the composition should be adjusted to provide the desired physical and handling properties before and after polymerization.

The dental composition or the respective pastes of the kit of parts may contain one or more fillers.

The nature and structure of the filler(s) is not particularly limited unless the intended purpose cannot be achieved.

Adding a filler can be beneficial e.g. for adjusting the rheological properties like the viscosity. The content of the filler also typically influences the physical properties of the composition after hardening, like hardness or flexural strength.

The size of the filler particles should be such that a homogeneous mixture with the hardenable component forming the resin matrix can be obtained—

The mean particle size of the filler may be in the range from 5 nm to 100 μm.

If desired, the measurement of the particle size of the filler particles can be done with a TEM (transmission electron microscopy) method, whereby a population is analysed to obtain an average particle diameter.

A preferred method for measuring the particle diameter can be described as follows: Samples approximately 80 nm thick are placed on 200 mesh copper grids with carbon stabilized form var substrates (SPI Supplies—a division of Structure Probe, Inc., West Chester, PA). A transmission electron microscopy (TEM) is taken, using JEOL™ 200CX (JEOL, Ltd. of Akishima, Japan and sold by JEOL USA, Inc.) at 200 Kv. A population size of about 50-100 particles can be measured and an average diameter is determined.

The filler(s) typically comprise non acid-reactive fillers. A non-acid reactive filler is a filler which does not undergo an acid/base reaction with an acid.

Useful non-acid reactive fillers include fumed silica, fillers based on non-acid reactive fluoroaluminosilicate glasses, quartz, ground glasses, non water-soluble fluorides such as CaF₂, silica gels such as silicic acid, in particular pyrogenic silicic acid and granulates thereof, cristobalite, calcium silicate, zirconium silicate, zeolites, including the molecular sieves.

Suitable fumed silicas include for example, products sold under the tradename Aerosil™ series OX-50, -130, -150, and -200, Aerosil™ R8200, —R805 available from Evonik, CAB-O-SIL™M5 available from Cabot Corp (Tuscola), and HDK types e.g. HDK™-H2000, HDK™ H15, HDK™ H18, HDK™ H20 and HDK™H30 available from Wacker.

Filler(s) which can also be used and which provide radiopacity to the dental materials described in the present text include heavy metal oxide(s) and fluoride(s). As used herein, “radiopacity” describes the ability of a hardened dental material to be distinguished from tooth structure using standard dental X-ray equipment in the conventional manner. Radiopacity in a dental material is advantageous in certain instances where X-rays are used to diagnose a dental condition. For example, a radiopaque material would allow the detection of secondary caries that may have formed in the tooth tissue surrounding a filling.

Oxides or fluorides of heavy metals having an atomic number greater than about 28 can be preferred. The heavy metal oxide or fluoride should be chosen such that undesirable colors or shading are not imparted to the hardened resin in which it is dispersed. For example, iron and cobalt would not be favoured, as they impart dark and contrasting colors to the neutral tooth color of the dental material. More preferably, the heavy metal oxide or fluoride is an oxide or fluoride of metals having an atomic number greater than 30. Suitable metal oxides are the oxides of yttrium, strontium, barium, zirconium, hafnium, niobium, tantalum, tungsten, bismuth, molybdenum, tin, zinc, lanthanide elements (i.e. elements having atomic numbers ranging from 57 to 71, inclusive), cerium and combinations thereof Suitable metal fluorides are e.g. yttrium trifluoride and ytterbium trifluoride. Most preferably, the oxides and fluorides of heavy metals having an atomic number greater than 30, but less than 72 are optionally included in the materials of the invention. Particularly preferred radiopacifying metal oxides include lanthanum oxide, zirconium oxide, yttrium oxide, ytterbium oxide, barium oxide, strontium oxide, cerium oxide, and combinations thereof The heavy metal oxide particles may be aggregated. If so, it is preferred that the aggregated particles are equal or less than 200 nm in average diameter.

Other suitable fillers to increase radiopacity are salts of barium and strontium especially strontium sulphate and barium sulphate.

Filler(s) which can also be used include nano-sized fillers such as nano-sized silica.

Suitable nano-sized particles typically have a mean particle size in the range of 5 to 80 nm.

Preferred nano-sized silicas are commercially available from Nalco Chemical Co. (Naperville, Ill.) under the product designation NALCO™ COLLOIDAL SILICAS (for example, preferred silica particles can be obtained from using NALCO™ products 1040, 1042, 1050, 1060, 2327 and 2329), Nissan Chemical America Company, Houston, Texas (for example, SNOWTEX-ZL, -OL, -O, -N, -C, -20L , -40, and -50); Admatechs Co., Ltd., Japan (for example, SX009-MIE, SX009-MIF, SC₁₀₅₀-MJM, and SC₁₀₅₀-MLV); Grace GmbH & Co. KG, Worms, Germany (for example, those available under the product designation LUDOX™, e.g., P-W50, P-W30, P-X30, P-T40 and P-T4OAS); Akzo Nobel Chemicals GmbH, Leverkusen, Germany (for example, those available under the product designation LEVASIL™, e.g., 50/50%, 100/45%, 200/30%, 200A/30%, 200/40%, 200A/40%, 300/30% and 500/15%), and Bayer MaterialScience AG, Leverkusen, Germany (for example, those available under the product designation DISPERCOLL™ S, e.g., 5005, 4510, 4020 and 3030).

Surface-treating the nano-sized silica particles before loading into the dental material can provide a more stable dispersion in the resin. Preferably, the surface-treatment stabilizes the nano-sized particles so that the particles will be well dispersed in the hardenable resin and results in a substantially homogeneous composition. Furthermore, it is preferred that the silica be modified over at least a portion of its surface with a surface treatment agent so that the stabilized particle can copolymerize or otherwise react with the hardenable resin during curing.

Thus, the silica particles as well as other suitable non acid-reactive fillers can be treated with a resin-compatibilizing surface treatment agent.

If present the filler(s) are typically present in the following amounts: lower amount: at least 1 or at least 5 or at least 10 wt. %; upper amount: utmost 80 or utmost 70 or utmost 60 wt. %; range: 1 to 80 or to 70 or 10 to 60 wt. %; wt. % with respect to the weight of the dental composition obtained if the Catalyst Paste and the Base Paste of the kit of parts were combined.

The dental composition or the respective pastes of the kit of parts may also include photo-initiator(s).

The photo-initiator may be present in the Base Paste or the Catalyst Paste or in both pastes. Typically, the photo-initiator is present in the Catalyst Paste.

The nature and structure of the photo-initiator is not particularly limited unless the intended purpose cannot be achieved. Suitable photo initiator(s) for free radical polymerization are generally known to the person skilled in the art dealing with dental materials.

As photo-initiator(s), those which can polymerize the polymerizable monomer(s) by the action of visible light having a wavelength of in the range of 350 nm to 500 nm are preferred.

Suitable photo-initiator(s) often contain an alpha di-keto moiety, an anthraquinone moiety, a thioxanthone moiety or benzoin moiety.

Examples of photo-initiator(s) include camphor quinone, 1-phenyl propane-1,2-dione, benzil, diacetyl, benzyl dimethyl ketal, benzyl diethyl ketal, benzyl di(2-methoxyethyl) ketal, 4,4,′-di-methylbenzyl dimethyl ketal, anthraquinone, 1-chloroanthraquinone, 2-chloroanthraquinone, 1,2-benz-anthraquinone, 1-hydroxyanthraquinone, 1-methylanthraquinone, 2-ethylanthraquinone, 1-bromoanthraquinone, thioxanthone, 2-isopropyl thioxanthone, 2-nitrothioxanthone, 2-methyl thioxanthone, 2,4-dimethyl thioxanthone, 2,4-diethyl thioxanthone, 2,4-diisopropyl thioxanthone, 2-chloro-7-trifluoromethyl thioxanthone, thioxanthone-10,10-dioxide, thioxanthone-10-oxide, benzoin methyl ether, benzoin ethyl ether, isopropyl ether, benzoin isobutyl ether, benzophenone, bis(4-dimethyl-aminophenyl)ketone, 4,4,′-bisdiethylaminobenzophenone.

Using acylphosphine oxides was found to be useful, as well.

Suitable acylphosphine oxides can be characterized by the following formula

(R⁹)₂—P(═O)—C(═O)—R¹⁰

wherein each R⁹ individually can be a hydrocarbyl group such as alkyl, cycloalkyl, aryl, and aralkyl, any of which can be substituted with a halo-, alkyl- or alkoxy-group, or the two R⁹ groups can be joined to form a ring along with the phosphorous atom, and wherein R¹⁰ is a hydrocarbyl group, an S-, O-, or N-containing five- or six-membered heterocyclic group, or a —Z—C(—O)—P(═O)—(R⁹)₂ group, wherein Z represents a divalent hydrocarbyl group such as alkylene or phenylene having 2 to 6 carbon atoms.

Suitable systems are also described e.g. in U.S. Pat. No. 4,737,593 (Ellrich et al.), the content of which is herewith incorporated by reference.

Preferred acylphosphine oxides are those in which the R⁹ and R¹⁰ groups are phenyl or lower alkyl- or lower alkoxy-substituted phenyl. By “lower alkyl” and “lower alkoxy” is meant such groups having from 1 to 4 carbon atoms. In particular, 2,4,6-trimethylbenzoyl diphenyl phosphine oxide was found to be useful (Lucirin™ TPO, BASF).

Suitable bisacylphosphine oxides can also be described by the following formula:

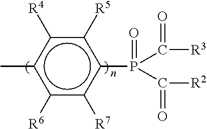

wherein n is 1 or 2, and R⁴, R⁵, R⁶ and R⁷ are H, C₁₋₄ alkyl, C₁₋₄ alkoxyl, F, Cl or Br; R² and R³, which are the same or different, stand for a cyclohexyl, cyclopentyl, phenyl, naphthyl, or biphenylyl radical, a cyclopentyl, cyclohexyl, phenyl, naphthyl, or biphenylyl radical substituted by F, Cl, Br, I, C1-4 alkyl and/or C₁₋₄ alkoxyl, or an S or N-containing 5-membered or 6-membered heterocyclic ring; or R² and R³ are joined to form a ring containing from 4 to 10 carbon atoms and being optionally substituted by 1 to 6 C₁₋₄ alkyl radicals.

More specific examples include: bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenyl-phosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dime thoxybenzoyl)-2,5-dimethylphenylphosphine oxide, bis-(2,6-dimethoxybenzoyl)-phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethylphenyl-phosphine oxide, bis-(2-methyl-1-naphthoyl)phenylphosphine oxide, bis-(2-methyl-1-naphthoyl)-4-biphenylylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5 -dimethylphosphine oxide, bis-(2-methoxy-1-naphthoyl)-4-ethoxyphenyl-phosphine oxide, bis-(2-methoxy-1-naphthoyl)-4-biphenylylphosphine oxide, bis-(2-methoxy-1-naphthoyl)-2-naphthylphosphine oxide and bis-(2-chloro-1-naphthoyl)-2,5-dime thylphenylphosphine oxide.

The acylphosphine oxide bis(2,4,6-trimethylbenzoyl)phenyl phosphine oxide (previously known as IRGACURE™ 819, Ciba Specialty Chemicals) is sometimes preferred.

If present, the photo-initiator is typically present in the following amounts: lower amount: at least or at least 0.2 or at least 0.3 wt. %; upper amount: up to 10 or up to 8 or up to 6 wt. %; range: 0.1 to 10 or 0.2 to 8 or 0.3 to 6 wt. %; wt. % with respect to the weight of the dental composition obtained if the Catalyst Paste and the Base Paste of the kit of parts were combined.

Examples of dyes or pigments, which can be used include titanium dioxide or zinc sulphide (lithopones), red iron oxide 3395, Bayferrox™ 920 Z Yellow, Neazopon™ Blue 807 (copper phthalocyanine-based dye) or Helio™ Fast Yellow ER. These additives may be used for individual colouring of the dental compositions.

Examples of photo-bleachable colorants which can be present include Rose Bengal, Methylene Violet, Methylene Blue, Fluorescein, Eosin Yellow, Eosin Y, Ethyl Eosin, Eosin bluish, Eosin B, Erythrosin B, Erythrosin Yellowish Blend, Toluidine Blue, 4′,5′-Dibromofluorescein and blends thereof. Further examples of photo-bleachable colorants can be found in U.S. Pat. No. 6,444,725.

Examples of fluoride release agents which can be present include naturally occurring or synthetic fluoride minerals. These fluoride sources can optionally be treated with surface treatment agents.

Further additives, which can be added, include stabilizers, especially free radical scavengers such as substituted and/or unsubstituted hydroxyaromatics (e.g. butylated hydroxytoluene (BHT), hydroquinone, hydroquinone monomethyl ether (MEHQ), 3,5-di-tert-butyl-4-hydroxyanisole (2,6-di-tert-butyl-4-ethoxyphenol), 2,6-di-tert-butyl-4-(dimethylamino)methylphenol or 2,5-di-tert-butyl hydroquinone, 2-(2′-hydroxy-5′-methylphenyl)-2H-benzotriazole, 2-(2′-hydroxy-5′-t-octylphenyl)-2H-benzotriazole, 2-hydroxy-4-methoxybenzophenone (UV-9), 2-(2′-hydroxy-4′,6′-di-tert-pentylphenyl)-2H-benzotriazole, 2-hydroxy-4-n-octoxybenzophenone, 2-(2′-hydroxy-5′-methacryloxyethylphenyl)-2H-benzotriazole, and phenothiazine.

Further additives, which can be added, include retarder(s), (such as 1,2-diphenylethylene), plasticizers (including polyethylene glycol derivatives, polypropylene glycols, low-molecular-weight polyesters, dibutyl, dioctyl, dinonyl and diphenyl phthalate, di(isononyl adipate), tricresyl phosphate, paraffin oils, glycerol triacetate, bisphenol A diacetate, ethoxylated bisphenol A diacetate, and silicone oils), and flavorant(s).

There is no need for these adjuvants or additives to be present, so adjuvants or additives might not be present at all. However, if they are present, they are typically present in an amount which is not detrimental to the intended purpose.

If present, additive(s) is (are) typically present in the following amounts: lower amount: at least wt. % or at least 0.05 wt. % or at least 0.1 wt. %; upper amount: utmost 15 wt. % or utmost 10 wt. % or utmost 5 wt. %; range: 0.01 wt. % to 15 wt. % or 0.05 wt. % to 10 wt. % or 0.1 wt. % to 5 wt. %, wt. % with respect to the weight of the dental composition obtained if the Catalyst Paste and the Base Paste of the kit of parts were combined.

The dental composition described in the present text may comprise, essentially consist of or consist of the respective components in the following amounts:
polymerizable components without acidic moiety: 5 to 65 wt. %,
polymerizable components with acidic moiety: 2 to 50 wt. %,
filler: 1 to 80 wt. %,
microcapsules comprising a first component of a redox-initiator system: 0.05 to 10 wt. %, second component of a redox-initiator system: 0.01 to 5 wt. %, additive(s): 0 to 10 wt. %,
wt. % with respect to the dental composition.

In another embodiment, the dental composition may comprise, essentially consist of or consist of the respective components in the following amounts:
polymerizable components without acidic moiety: 20 to 65 wt. %,
polymerizable components with acidic moiety: 2 to 20 wt. %,
filler: 30 to 70 wt. %,
microcapsules comprising a first component of a redox-initiator system: 0.1 to 5 wt. %,
second component of a redox-initiator system: 0.01 to 5 wt. %,
additive(s): 0 to 10 wt. %,
wt. % with respect to the dental composition.

In order to avoid an undesired reaction, acid and basic components should not be stored together during storage, in particular together with the microcapsules comprising the pH-sensitive component.

The microcapsules should also not be stored together with water. Thus, if the dental composition is provided as a kit of parts comprising a catalyst and a base paste, the paste comprising the microcapsules should be essentially free of water.

Further, to reduce the risk of conflicting reactions, it can be advantageous if the dental compositions is essentially free of or does not contain additional pH-sensitive or acid-reactive materials such as calcium carbonate fillers.

The dental composition described in the present text is typically produced by combining or mixing the respective components, i.e. the polymerizable components and the microcapsules described in the present text together with other optional components, such a filler(s), or additives.

Mixing also includes kneading. If desired, a speed mixer can be used. Depending on the components to be mixed, the mixing is done under save light conditions.

The dental composition described in the present text is typically stored in a packaging device.

If the dental composition is provided as a kit of parts comprising a Catalyst Paste and a Base Paste, the pastes may be contained in separate sealable vessels (e.g. made out of plastic or glass).

For use, the practitioner may take adequate portions of the compositions contained from the vessels and mix the portions by hand on a mixing plate.

According to a preferred embodiment, the Catalyst Paste and the Base Paste are contained in separate compartments of a storing device.

The storing device typically comprises two compartments for storing the respective parts, each compartment being equipped with a nozzle for delivering the respective part. Once delivered in adequate portions, the parts can then be mixed by hand on a mixing plate.

According to another preferred embodiment, the storing device has an interface for receiving a static mixing tip. The mixing tip is used for mixing the respective pastes. Static mixing tips are commercially available e.g. from SulzerMixpac company.

Suitable storing devices include cartridges, syringes and tubes.

The storing device typically comprises two housings or compartments having a front end with a nozzle and a rear end and at least one piston movable in the housing or compartment.

Cartridges which can be used are described e.g. in US 2007/0090079 A1 (Keller) or US (Keller et al.), the disclosure of which is incorporated by reference. Some of the cartridges which can be used are commercially available e.g. from Sulzer Mixpac AG (Switzerland). Static mixing tips which can be used are described e.g. in US 2006/0187752 A1 (Keller) or in U.S. Pat. No. 5,944,419 (Streiff), the disclosure of which is incorporated by reference. Mixing tips which can be used are commercially available from Sulzer Mixpac AG (Switzerland), as well.

Other suitable storing devices are described e.g. in WO 2010/123800 (3M), WO 2005/016783 (3M), WO 2007/104037 (3M), WO 2009/061884 (3M), in particular the device shown in FIG. 14 of WO 2009/061884 (3M) or WO 2015/073246 (3M), in particular the device shown in FIG. 1 of WO 2015/07346. Those storing devices have the shape of a syringe. The content of these references is herewith incorporated by reference, as well.

Alternatively, but less preferred, paste/paste compositions described in the present text can be provided in two individual syringes and the individual pastes can be mixed by hand prior to use.

Thus, the invention is also directed to a device for storing the kit of parts described in the present text, the device comprising two compartments, Compartment A and Compartment B, Compartment A containing the Catalyst Paste and Compartment B containing the Base Paste, the Catalyst Paste and the Base Paste being as described in the present text, Compartment A and Compartment B both comprising a nozzle or an interface for receiving an entrance orifice of a static mixing tip.

The mixing ratio of the Base Paste and the Catalyst Base Paste is typically 3:1 to 1:3 with respect to volume, preferably 2:1 to 1:2, more preferably 1:1.

The microcapsule described in the present text is particularly useful for producing a curable composition comprising curable components and a redox-initiator system.

According to one embodiment, the curable composition is a dental or orthodontic composition.

According to one embodiment, the curable composition is a dental or orthodontic cement, adhesive or filing material.

The microcapsule described in the present text are particularly useful for producing a curable composition obtained by combining two pastes, a base paste and a catalyst paste, wherein one of the pastes contain the microcapsules described in the present text and the other paste contains an acidic component.

When mixing the two pastes, the paste containing the acidic component comes in contact with the microcapsules. Upon contact, the microcapsule is weakened. This enables the component of the redox-initiator system to be released from the microcapsule more easily.

As self-adhesive dental materials usually contain an acidic paste, the acidity of this paste can be used as trigger to weaken the microcapsule.

The invention also relates to a process of curing a dental curable composition.

Such a process comprises the following steps: A Catalyst Paste comprising the microcapsules described in the present text and a Base Paste comprising an acidic or basic component is provided.

The microcapsules contain as component to be released a first component of a redox-initiator system.

Either the Catalyst Paste or the Base Paste or the Catalyst Paste and the Base Paste comprise curable components and a second component of the redox-initiator system.

The first and second component of the redox-initiator system forming an initiator system are able to initiate the curing of the curable components.

The Catalyst Paste and the Base Paste are mixed.

The acidic component contained in the Base Paste dissolves or weakens the microcapsule resulting in a release of the redox-initiator component contained therein.

If brought in contact with each other, the redox-initiator components initiate the curing of the curable components of the curable composition.

The invention is also directed to a dental composition as described in the present text for use in a method of restoring a dental tooth in the mouth of a patient, the method comprising the steps of providing the dental composition in the form of a kit of parts comprising a Base Paste and a

Catalyst Paste as described in the present text,
mixing the Base Paste and the Catalyst Paste,
applying the mixture to the surface of a prepared tooth to be restored.

Further suitable embodiments are described below:

According to one embodiment, the kit of parts is characterized as follows:
the Catalyst Paste comprising
   the microcapsules described in the present text comprising a reducing component, preferably an ascorbic acid component,
   curable non-acidic (meth)acrylate component(s),
   filler(s),
      the Base Paste comprising
acidic component(s), preferably a polymerizable component comprising an acidic moiety,
curable (meth)acrylate component(s),
filler(s),
oxidizing component,
the reducing component and the oxidizing agent forming a redox-initiator system for curing the curable (meth)acrylate component(s).

According to another embodiment, the kit of parts is characterized as follows:

the Catalyst Paste comprising
the microcapsules described in the present text comprising an oxidizing component, preferably a component comprising a peroxide moiety,
curable non-acidic (meth)acrylate component(s), filler(s),
   the Base Paste comprising
acidic component(s), preferably a polymerizable component comprising an acidic moiety,
curable (meth)acrylate component(s),
filler(s),
reducing component,
   the reducing component and the oxidizing agent forming a redox-initiator system for curing the curable (meth)acrylate component(s).

The invention is also directed to a kit of parts comprising the dental composition described in the present text and the following parts alone or in combination:
a dental milling block for machining a dental restoration,
a dental adhesive.

Suitable dental milling blocks typically comprise a porous zirconia material, which contains yttria as a phase-stabilizing component and colouring components. Examples of dental milling blocks are described in US 2017/020639 (Jahns et al.), U.S. Pat. No. 2015/238291 A1 (Hauptmann et al.).

Suitable dental adhesives are acidic dental composition with a rather low viscosity (e.g. 0.01 to 3 Pa*s at 23° C.). Dental adhesives directly interact with the enamel or dentin surface of a tooth. Dental adhesives are typically one-part compositions, are radiation-curable and comprise ethylenically unsaturated component(s) with acidic moiety, ethylenically unsaturated component(s) without acidic moiety, water, sensitizing agent(s), reducing agent(s) and additive(s). Examples of dental adhesives are described in US 2020/0069532 A1 (Thalacker et al.) and U.S. 2017/0065495 A1 (Eckert et al.), U.S. 2019/231494 A1 (Dittmann et al.),

Thus, the kit of parts contains parts or components which can be used together in a process for restoring a defect tooth.

The dental milling block is used for machining a dental restoration, the dental adhesive is used for treating the surface of tooth to be restored and the dental composition described in the present text is used for cementing the dental restoration machined from the dental mill block.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. The above specification, examples and data provide a description of the manufacture and use of the compositions and methods of the invention. The invention is not limited to the embodiments disclosed herein. One skilled in the art will appreciate that many alternative embodiments of the invention can be made without departing from the spirit and scope of thereof.

The following examples are given to illustrate the invention.

### EXAMPLES

Unless otherwise indicated, all parts and percentages are on a weight basis, all water is de-ionized water, and all molecular weights are weight average molecular weight. Moreover, unless otherwise indicated all experiments were conducted at ambient conditions (23° C.; 1013 mbar).

### Methods

### Scanning Electron Microscopy (SEM)

If desired, the particle size and the shape of the microcapsules can be further analysed and determined by SEM, e.g. using the device JSM 5400 (Hitachi).

### Light-Scattering

If desired, the particle size distribution can be determined by light-scattering, e.g. using the device Horiba (Horiba, JP). The light scattering particle-sizer illuminates the sample with a laser and analyzes the intensity fluctuations of the light scattered from the particles at an angle of 173 degrees. The method of Photon Correlation Spectroscopy (PCS) can be used by the instrument to calculate the particle size. PCS uses the fluctuating light intensity to measure Brownian motion of the particles in the liquid. The particle size is then calculated to be the diameter of sphere that moves at the measured speed. The intensity of the light scattered by the particle is proportional to the sixth power of the particle diameter. The Z-average size or cumulant mean is a mean calculated from the intensity distribution and the calculation is based on assumptions that the particles are mono-modal, mono-disperse, and spherical. Related functions calculated from the fluctuating light intensity are the Intensity Distribution and its mean. The mean of the Intensity Distribution is calculated based on the assumption that the particles are spherical. Both the Z-average size and the Intensity Distribution mean are more sensitive to larger particles than smaller ones.

The volume distribution gives the percentage of the total volume of particles corresponding to particles in a given size range. The volume-average size is the size of a particle that corresponds to the mean of the Volume Distribution. Since the volume of a particle is proportional to the third power of the diameter, this distribution is less sensitive to larger particles than the Z-average size. Thus, the volume-average will typically be a smaller value than the Z-average size. In the scope of this document the Z-average size is referred to as “mean particle size”.

### pH Value

If desired, the pH value of can be determined as follows: 1.0 g of a component (e.g. filler) is dispersed in 10 ml de-ionized water and stirred for about 5 min. A calibrated pH electrode is dipped into the suspension and the pH value is determined during stirring.

### Elemental Composition

If desired, the elemental composition can be determined by X-ray fluorescence spectrometry (XRF), e.g. with the ZSX Primus II from Rigaku, Japan. This method is especially suited for the analysis of solids, e.g. zirconia ceramics or glass materials.

### Mechanical Stability

If desired, the mechanical stability of the microcapsules can be determined as follows: The microcapsules to be analysed are filled with Sudan blue II (a dye having a blue color). Pastes are then prepared using e.g. the following composition: 18 wt. % TEGDMA, 20 wt. % UDMA, 52.02 wt. % glass filler, 8.0 wt. % fumed silica, 0.1 wt. % IC 819, 0.46 wt. % coated filled microcapsules. The composition is mixed by using a commercially available speed mixer (e.g. SpeedMixer™ DAC 150 SP; Hauschild, Germany) applying the following conditions: 3×90 s, 2500 RPM and 3×20 s 3500 RPM with cooling to room temperature after each mixing step.

From this composition light-cured discs are prepared: 700 mg of the paste is filled into a cylindrical mold (15 mm diameter; 1.5 mm height) which is placed between glass slides covered with transparency films. This sandwich-like structure is irradiated from both sides for 20 s (without light guide) using an Elipar™ S10 light curing device (3M Oral Care).

Then, the specimen is removed from the mold and put into a Visio™ Beta Vario light oven with vacuum (3M Oral Care) for 7 min to fully light cure the sample.

Then, the L*a*b* color coordinates are determined. If the b* value is positive, the blue color Sudan Blue II obviously did not release from the microcapsules during the paste and disc preparation. This is an indication that the tested microcapsules are mechanically stable. If the b* value is negative, the blue color Sudan Blue II obviously did release from the microcapsules during the paste and disc preparation. This is an indication that the tested microcapsules are mechanically not stable.

Water Solubility

If desired, the water-solubility of a substance can be determined by adding 0.1 g of the component to be tested to 100 g water (23° C.) and stirring the composition for 10 min. If the component is completely dissolved, the water solubility is at least 0.1 g per 100 ml water.

### Determination of Working & Setting Time

A Physica MCR 301 Rheometer (Anton Paar, Graz, Austria) with a plate/plate geometry of 8 mm diameter at a temperature of 28° C. was used. 100 mg of Paste AX was hand mixed with 100 mg of Paste B1 (mixing ratio 1.0:1.0 w/w) for 20 s on a mixing pad with the help of a spatula. Then, the mixture was applied between the plates and the gap was set to 0.75 mm. Frequency was 1.25 Hz, oscillation with 1.75% deflection.

The working time (Ta) is defined as the time between start of mixing and time of reaching intersection point of G′ and G″. The setting time (Tf) is defined as the time between start of mixing and the time for the mixed pastes to reach a shear stress of 100,000 Pa.

### Materials

TABLE 1

| Compound | Abbreviation |
|---|---|
| Sodium bicarbonate | |
| Sodium carbonate | |
| Calcium acetate | |
| APG 325 (BASF), nonionic alkyl polyglucoside surfactant | SURF |
| Triethylene glycol dimethacrylate | TEGDMA |
| Diurethane dimethacrylate | UDMA |
| Hydroxyethyl methacrylate | HEMA |
| 1,3-glycerol dimethacrylate phosphate | GDP |
| De-ionized water | H₂O |
| Glass powder G018-163 (Schott ™), 2.5% silanized | GP |
| OX 50 filler (Evonik ™), 3.0% silanized | OX50 |
| Aerosil ® R805 (Evonik ™) | R805 |
| Aerosil ® 200 (Evonik ™) | Aerosil 200 |
| Ascorbyl palmitate coated with CaCO₃ | MC-ASP |
| Ascorbyl palmitate | ASP |
| Triphenyl phosphite | TPP |
| Amyl hydroperoxide | AHP |
| 2 wt. % Amine HCl in TEGDMA | Amine-HCl |
| BHT, Cu(Ac)₂ in GDP | Solution 1 |
| Irgacure ™ 819 | IC 819 |

### Method of Coating Ascorbyl Palmitate Particles:

To 200 g distilled water were added: 10 g sodium bicarbonate (alternatively potassium bicarbonate, ammonium bicarbonate or any other monovalent cation bicarbonate), 20 g sodium carbonate (alternatively potassium carbonate, ammonium carbonate or any other monovalent cation hydrogen carbonate), 200 mg of SURF, 10 g of ASP.

The mixture was blended using a high shear rotary blender (IKA T50 Ultra Turrax™) for 3 minutes. The blended mixture was kept for 15 min at 20° C. The mixture was then centrifugated at 4,000 RPM for 3 min. The supernatant was discarded and the sedimented part was transferred to 50 g 28% aqueous solution of calcium acetate. The mixture was then blended for about 3 min (mixing device: IKA T50 Ultra Turrax). The blended dispersion was then left to stand for about 15 min at room temperature (20° C.). This was then centrifugated at 4,000 rpm for 3 min. The supernatant was discarded. The sedimented part was re-suspended in distilled water and filtered. The resulting solids were air dried then vacuum dried.

### Compositions

All Pastes AX were prepared by weighing in the respective compounds.

The compositions of the pastes given in Table 2 were mixed by using a commercially available SpeedMixer™ DAC 150 SP (Hauschild, Germany) by application of 3×90 s, 2500 RPM and 3×20 s 3500 RPM (cooling to room temperature after each mixing step).

TABLE 2

| Compound | Paste A1 | Paste A2 |
|---|---|---|
| TEGDMA | 18.0 | 18.0 |
| UDMA | 20.0 | 20.0 |
| GP | 51.0 | 50.4 |
| R805 | 8.0 | 8.0 |
| ASP | 0.4 | — |
| MC-ASP | — | 1.0 |
| TPP | 0.5 | 0.5 |
| 2 wt. % Amine-HCl in TEGDMA | 2.0 | 2.0 |
| IC 819 | 0.1 | 0.1 |

Paste B1 was prepared by weighing in the respective compounds given in Table 3. Then, the mixture was mixed by using a commercially available SpeedMixer™ DAC 150 SP (Hauschild, Germany) by application of 3×90 s, 2500 RPM and 2×60 s 3500 RPM (cooling to room temperature after each mixing step).

TABLE 3

| | Paste B1 |
|---|---|
| Compound | Amount [wt. %] |
| HEMA | 20.0 |
| GDP | 15.0 |
| H₂O | 10.0 |
| OX50 | 45.2 |
| Aerosil 200 | 1.5 |
| Solution 1 | 6.0 |
| AHP | 2.3 |

Different kits of parts were provided (Examples 1-12) as shown in Table 4. Paste A1 and A2 were stored at room temperature (23° C.), 36° C. or 50° C. for the storage time listed. Paste B1 was stored at room temperature (23° C.).

TABLE 4

| | | | Pastes A1, A2 | Paste B | Storage |
|---|---|---|---|---|---|
| | | | Storage at | Storage at | Time |
| Example | Paste | Paste | [° C.] | [° C.] | [days] |
| 1 | A1 | B1 | 23 | 23 | 0 |
| 2 | A1 | B1 | 23 | 23 | 1 |
| 3 | A1 | B1 | 36 | 23 | 1 |
| 4 | A1 | B1 | 50 | 23 | 1 |
| 5 | A1 | B1 | 36 | 23 | 4 |
| 6 | A1 | B1 | 50 | 23 | 4 |
| 7 | A2 | B1 | 23 | 23 | 0 |
| 8 | A2 | B1 | 23 | 23 | 1 |
| 9 | A2 | B1 | 36 | 23 | 1 |
| 10 | A2 | B1 | 50 | 23 | 1 |
| 11 | A2 | B1 | 36 | 23 | 6 |
| 12 | A2 | B1 | 50 | 23 | 6 |

Working and setting time of the compositions of Examples 1-12 were determined in dependency on storage time and temperature as described above. The results are given in Table 5.

TABLE 5

| | ASP in | Storage | Storage Temp. of | Working | Setting |
|---|---|---|---|---|---|
| Exam- | Composition | Time | Pastes A1 or A2 | Time | Time |
| ple | [wt. %] | [days] | [° C.] | [min] | [min] |
| 1 | 0.20 | 0 | 23 | 2.4 | 3.2 |
| 2 | 0.20 | 1 | 23 | 2.4 | 3.1 |
| 3 | 0.20 | 1 | 36 | 2.2 | 2.9 |
| 4 | 0.20 | 1 | 50 | 2.2 | 2.9 |
| 5 | 0.20 | 4 | 36 | 2.4 | 3.2 |
| 6 | 0.20 | 4 | 50 | 15.0 | >30.0 |
| 7 | 0.20 | 0 | 23 | 2.3 | 3.1 |
| 8 | 0.20 | 1 | 23 | 2.3 | 3.1 |
| 9 | 0.20 | 1 | 36 | 2.4 | 3.3 |
| 10 | 0.20 | 1 | 50 | 2.4 | 3.2 |
| 11 | 0.20 | 6 | 36 | 2.7 | 3.9 |
| 12 | 0.20 | 6 | 50 | 2.9 | 4.4 |

The compositions obtained after mixing Pastes A 1 and A2 with Paste B1 showed very similar working and setting times in the desired range (Example 1 and 7).

Paste A1 containing a non water-soluble component of a redox-initiator system showed a reduced working and setting time after 4 weeks at 50° C. (Example 6), indicating some degradation of the component of the redox-initiator system.

Paste A2 containing an encapsulated non water-soluble component of a redox-initiator system showed only a slightly reduced working and setting time even after 6 weeks at 50° C. (Example 12).

Thus, using microcapsules as described in the present text are not only sufficiently stable to survive mixing processes, but also help to increase the shelf-life stability of redox-initiator components in pastes.

Further, it was found that the working and setting time of a curable composition containing redox-initiator components which are encapsulated in a microcapsule comprising a crosslinked polymeric material may vary to a broader extent which may result in less predictable curing behavior.

Compared to microcapsules described e.g. in US 2020/368117 A1 (Claussen et al.), the microcapsules described in the present text allow for a higher loading of the first component of the redoxinitiator system. If desired, the loading capacity can be increased by a factor up to about 5 or 4 or 3 by weight. Thus, for providing a dental composition with the same amount of redox-initiator system lower amounts of microcapsules are needed.

1. A dental composition comprising
a polymerizable component not comprising an acidic moiety, and microcapsules comprising
a non water-soluble first component of a redox-initiator system, and
a pH-sensitive inorganic component,
the microcapsules not comprising a polymeric material,
the pH-sensitive inorganic component preferably forming a shell around the core which is comprised of the non-water soluble first component.

2. The dental composition according to claim 1, the pH-sensitive inorganic component comprising an anion and a multivalent cation, the
the anion being preferably selected from carbonate, hydrogen carbonate, phosphate, nitrate and sulphate,
the multivalent cation being preferably selected from ions of Mg, Ca, Sr, Ba, Al or Zn.

3. The dental composition according to claim 1, the pH-sensitive inorganic component being able to produce gas upon contact with an acidic component, the pH-sensitive component being preferably selected from carbonates and hydrogen carbonates of multivalent cations.

4. The dental composition according to claim 1, the non water-soluble first component of the redox-initiator system being a reducing agent.

5. The dental composition according to claim 1, the ratio of the pH-sensitive inorganic component of the microcapsules to the non water-soluble first component of the redox-initiator system being in a range of 20 to 1 to 1 to 1 with respect to weight.

6. The dental composition according to claim 1, the microcapsules having a diameter of 1 to 200 μm.

7. The dental composition according to claim 1, the dental composition being provided as a kit of parts comprising a Catalyst Paste and a Base Paste, the Catalyst Paste comprising
a polymerizable component not comprising an acidic moiety,
the microcapsules comprising a non water-soluble first component of a redox-initiator system,
optionally filler,
the Base Paste comprising
an acidic component, preferably a polymerizable component comprising an acidic moiety,
a second component of the redox initiator system,
optionally filler.

8. The dental composition according to claim 1 the dental composition being provided as a kit of parts comprising a Catalyst Paste and a Base Paste, the Catalyst Paste comprising
a polymerizable component not comprising an acidic moiety,
filler,
microcapsules comprising a pH-sensitive component and a non water-soluble first component of a redox-initiator system,
the pH-sensitive component comprising the salt of a multivalent cation and a carbonate or hydrogen carbonate anion,
the non water-soluble first component of a redox-initiator system being a non water-soluble ascorbic acid component,
the microcapsule having a diameter in the range of 1 to 100 μm,
the Base Paste comprising
a polymerizable component comprising an acidic moiety, filler,
a second component of the redox-initiator system, the second component of the redox-initiator system being selected from oxidizing components and transition metal components.

9. The dental composition according to claim 7, the acidic component being characterized by the following features alone or in combination:
pKs value: below 5;
comprising an acidic moiety selected from a sulfonic, sulfinic, phosphoric, phosphonic, phosphinic, or carboxylic moiety.

10. The dental composition according to claim 1, the dental composition being a self-adhesive dental cement composition.

11-12. (canceled)

13. A device for storing the dental composition described in claim 7, the device comprising two compartments, Compartment A and Compartment B, Compartment A containing the Catalyst Paste and Compartment B containing the Base Paste, the Catalyst Paste and the Base Paste being as described in the preceding claims, Compartment A and Compartment B both comprising a nozzle or an interface for receiving an entrance orifice of a static mixing tip.

14. A kit of parts comprising the dental composition according to claim 1, and the following parts alone or in combination: a dental milling block for machining a dental restoration; a dental adhesive.

15. (canceled)
